(19) United States
(12) Patent Application Publication
(10) Pub. No.: US 2026/0126423 A1
(43) Pub. Date: May 7, 2026

(54) SYSTEM FOR MEASURING AND TRANSMITTING A VARIABLE DIHYDROGEN CONTENT FOR AN EXTERNAL RECEIVER

(71) Applicant: TotalEnergies OneTech, Courbevoie (FR)

(72) Inventors: Nicolas Obrecht, Solaize (FR); Cyrille Callu, Solaize (FR)

(21) Appl. No.: 18/861,088

(22) PCT Filed: Apr. 28, 2023
(86) PCT No.: PCT/EP2023/061316

§ 371 Date: Jul. 18, 2025

(30) Foreign Application Priority Data
Apr. 28, 2022 (FR) FR2203995

(51) Int. Cl. G01N 33/00

(52) U.S. Cl. CPC G01N 33/005; G01N 33/0073

(57) ABSTRACT

The present invention relates to a system for real-time measurement of a variable dihydrogen content 3 in a gas stream and for transmitting information relating to said measured dihydrogen content in the gas stream to at least one external receiver 4 located outside said system and not communicating with a combustion system.

The invention also relates to a method for real-time measurement of the dihydrogen content in a gas stream and for transmitting information representative of said dihydrogen content to at least one receiver 4 not communicating with a combustion system.

The present invention relates to a system for real-time measurement of a variable dihydrogen content present in a gas stream and for transmitting information representative of said measured dihydrogen content in the gas stream to at least one external receiver different from a dihydrogen burner.

The invention also relates to a method for real-time measurement of a variable dihydrogen content present in a gas stream and for transmitting information representative of said measured dihydrogen content in the gas stream to at least one external receiver different from a dihydrogen burner.

The invention also relates to a method for optimising a method for producing dihydrogen, preferably chosen in the group consisting of natural gas decomposition, ammonia cracking, natural gas steam reforming or water electrolysis, or a method for separating a mixture of natural gas and dihydrogen, in particular a membrane or electrolysis separation method.

The invention also relates to an assembly comprising at least one system for producing a gas stream comprising a variable dihydrogen content, connected to at least one system for real-time measurement of a variable dihydrogen content present in the gas stream and for transmitting information representative of said measured dihydrogen content to at least one system different from a dihydrogen burner.

The use of dihydrogen, for example from decarbonised or renewable sources, in particular in natural gas-based mixtures, appears to represent at the present time an advantageous and promising solution in order to reduce greenhouse gas emissions in numerous applications.

By way of illustration, mention may in particular be made of electricity production, by isolated-site gas turbine or in CCGT type thermal power plants, industrial boilers, in particular hydrogen wall-mounted boilers, or natural gas motors frequently used for road transport or construction equipment, but mention may be made more generally of industrial methods controlled thanks to dihydrogen obtained with a production or fractionation method, for example by cracking ammonia.

Dihydrogen also has the advantage of being capable of being injected directly into current natural gas transport, distribution or storage networks which makes it possible to route hybrid mixtures based on natural gas and dihydrogen without necessarily incurring additional high investment costs on setting up specialised infrastructures.

Dihydrogen may be obtained by decomposing natural gas or ammonia, by natural gas steam reforming with optionally a step of capturing carbon dioxide, by coal or biomass gasification, or methane pyrolysis, or by decomposing water, in particular by electrolysis.

Dihydrogen may also be obtained by separating dihydrogen mixed with natural gas, in particular thanks to the use of a membrane or electrochemical separation method in a mixture based on natural gas and dihydrogen.

However, the performances of the gas streams obtained thanks to the use of these methods are found to depend directly on their point-in-time dihydrogen content, i.e. the conversion rate of the initial gas or liquid stream into dihydrogen.

Indeed, this conversion rate generally represents an important factor to take into consideration during the use of industrial methods controlled by a dihydrogen production method.

In other words, the technical, or environmental, performances of systems controlled via a method for producing or separating dihydrogen, in particular within the scope of the applications mentioned above, may be impacted by the point-in-time dihydrogen content.

Hence, it may be advantageous to monitor and control parameters aimed at influencing the transformation rate of an initial gas or liquid stream into dihydrogen, for example temperature, pressure conditions, electric field, etc., in order to best optimise the dihydrogen content obtained and result in gas streams more enriched in dihydrogen.

In order to better control the performances associated with dihydrogen-enriched gas streams, the solutions proposed at the present time consist for example of using gas streams having a fixed mean dihydrogen concentration according to the desired industrial application.

Under such conditions, systems controlled via a method for producing dihydrogen or a method for separating a mixture based on natural gas and dihydrogen are not capable of making full use of the benefits provided by the use of dihydrogen.

In light of the above, one of the aims of the present invention is that of implementing a system capable of monitoring precisely in real time the variable dihydrogen content at a given time in an initial liquid or gas stream, in particular in order to best optimise process control associated with hydrogen production or separation.

In other words, there is a real need to monitor and control one or more conversion parameters of an initial liquid or gas stream into dihydrogen in order to best optimise the dihydrogen content obtained and result in streams more enriched in dihydrogen.

Therefore, the present invention relates to a system for real-time measurement of the dihydrogen content present in a gas stream and for transmitting information representative of said dihydrogen content to at least one external receiver located outside said system; said system comprising:
at least one unit for measuring the dihydrogen content contained in the gas stream passing through said system,
at least one unit for processing and transmitting the information representative of said dihydrogen content determined by said measurement unit to at least one external receiver located outside said system not burning dihydrogen.

The system according to the invention thus makes it possible to determine and monitor in real time the point-in-time dihydrogen content present in a gas stream and communicate in real time the information representative of this content to at least one system not burning dihydrogen, i.e. not using dihydrogen or a gas stream containing dihydrogen as a fuel.

In other words, the external receiver is in particular different from a combustion system or a unit intended to monitor one or more combustion parameters of a gas stream containing dihydrogen.

Thus, the external receiver located outside the system not burning dihydrogen does not communicate with a combustion system.

The external receiver corresponds to a system for controlling a unit for producing dihydrogen or a unit for separating dihydrogen in a mixture based on natural gas and dihydrogen.

The system according to the invention makes it possible in particular to measure in real time the dihydrogen content (H₂) in a gas stream, at the outlet of a system for producing dihydrogen (or a system for producing a dihydrogen-enriched gas stream), and for transmitting the information representative of said measured H₂ content to a system capable of controlling one or more operating parameters of the system for producing dihydrogen via a closed loop based on said dihydrogen (H₂) content information.

The system according to the invention therefore has the advantage of improving the conversion of an initial liquid or gas stream at the inlet of a system for producing dihydrogen (or a system for producing a dihydrogen-enriched gas stream), in particular by enabling the control of one or more conversion parameters of the initial dihydrogen stream thanks to the H₂ content information transmitted in real time.

Thus, thanks to the information transmitted in real time on the H₂ content, the system according to the invention may be used in a closed loop to allow the control of one or more parameters, such as the temperature, pressure or electric field, implemented during a method for producing dihydrogen in order to improve the dihydrogen content.

The system according to the invention thus makes it possible to best optimise the dihydrogen content obtained at the outlet of a system for producing a gas stream containing a variable dihydrogen content.

In other words, the system according to the invention makes it possible to result in gas streams more enriched in dihydrogen.

The system according to the invention may therefore be advantageously used in a closed control loop in order to control one or more parameters of a method for producing dihydrogen to obtain gas flows more enriched in dihydrogen.

In particular, the system according to the invention makes it possible to measure the actual dihydrogen content present in the gas stream, at the outlet of a method for producing dihydrogen, and to transmit the corresponding information to a system capable of controlling in a closed loop one or more parameters of the method for producing dihydrogen which makes it possible to maximise the dihydrogen concentration according to the transmitted information.

Moreover, the system according to the invention also makes it possible to measure and communicate in real time the dihydrogen content obtained in particular from a decarbonised source, for example obtained from a water electrolysis method or an ammonia cracking method, or obtained from a carbon source, for example thanks to a methane pyrolysis or natural gas steam reforming method during which emitted carbon dioxide is optionally captured.

Preferably, the system according to the invention makes it possible to make full use of dihydrogen obtained from a decarbonised source.

The system according to the invention also makes it possible to measure and communicate in real time the dihydrogen content obtained from a separation method in a mixture of natural gas and dihydrogen, for example via a membrane or electrochemical separation method.

The system according to the invention thus has the advantage of allowing the control of systems for producing dihydrogen, for example by decomposing natural gas or cracking ammonia, or systems for separating dihydrogen and natural gas by membrane or electrolysis.

The system according to the invention thus makes it possible to optimise the control of such methods in real time.

In particular, the system according to the invention makes it possible to quantify dihydrogen drawn from a natural gas and dihydrogen network in order to supply a dihydrogen distribution station.

Furthermore, the system according to the invention may also report in real time the dihydrogen quality of a hybrid fuel mixture and the possible presence of dihydrogen leaks during the mixture routing process.

Thus, the system according to the invention makes it possible to report in real time any fluctuation of the dihydrogen content in a hybrid mixture.

The present invention also relates to the use of a system as described above for determining the dihydrogen content present in a gas stream and for transmitting information representative of said dihydrogen content to at least one receiver, located outside said system, not burning dihydrogen.

Preferably, the system according to the invention is used in a dihydrogen production unit, in a natural gas station supplied by at least one dihydrogen production unit.

The external receiver is as defined above and corresponds in particular to a system for controlling a unit for producing dihydrogen or a unit for separating dihydrogen in a mixture based on natural gas and dihydrogen.

Similarly, the invention also relates to a method for real-time measurement of the dihydrogen content in a gas stream and for transmitting information representative of said dihydrogen content to at least one receiver not burning dihydrogen, comprising:
a step of distributing a gas stream comprising a variable dihydrogen content, obtained by a unit for producing dihydrogen or a unit for separating a mixture of natural gas and dihydrogen,
a step of measuring the variable dihydrogen content present in the gas stream from the production step,
a step of processing and transmitting the information representative of said dihydrogen content, determined during the measurement step, at least one receiver not burning dihydrogen.

The external receiver is as defined above and corresponds in particular to a system for controlling a unit for producing dihydrogen or a unit for separating dihydrogen in a mixture based on natural gas and dihydrogen.

Advantageously, the method according to the invention comprises a step of controlling the step of producing the gas stream comprising a variable dihydrogen content or the step of separating a mixture of natural gas and dihydrogen according to the information representative of the dihydrogen content transmitted during the processing and transmission step.

Thus, the method according to the invention is preferably a method for controlling a unit for producing dihydrogen or a unit for separating a mixture of natural gas and dihydrogen using the measurement and transmission system according to the invention.

Thus, the method according to the invention is advantageously a method for optimising a method for producing dihydrogen chosen in the group consisting of natural gas decomposition, natural gas steam reforming, ammonia cracking, water electrolysis, or a method for separating a mixture of natural gas and dihydrogen.

The optimisation method thus implemented makes it possible to maximise the dihydrogen content obtained in a gas stream at the outlet of a production or separation method as described above.

The invention further relates to an assembly comprising at least one system for producing a gas stream comprising a variable dihydrogen content, connected to at least one measurement and transmission system, and at least one system different from a dihydrogen burner.

Other features, aspects and advantages of the invention will appear more clearly upon reading the following description and examples.

Hereinafter, and unless stated otherwise, the bounds of a range of values are comprised within this range.

The expression “at least one” is equivalent to the expression “one or more”.

### Measurement and Transmission System

As stated above, the measurement and transmission system according to the invention comprises:
at least one unit for measuring the dihydrogen content present in the gas stream passing through said system,
at least one unit for processing and transmitting the information representative of said dihydrogen content determined by said measurement unit to at least one external receiver located outside said system not burning dihydrogen.

The external receiver is as defined above.

Thus, the measurement unit determines in real time the dihydrogen content present in a gas stream, preferably in a gas stream obtained at the outlet of a method for producing dihydrogen or a method for separating dihydrogen in a mixture based on natural gas and dihydrogen.

Preferably, the measurement unit determines in real time the dihydrogen content present in a gas stream from a unit for producing dihydrogen by natural gas or ammonia decomposition, or by natural gas steam reforming, or coal or biomass gasification, or from a unit for separating dihydrogen from a mixture based on natural gas and dihydrogen.

According to the present invention, the gas stream comprises a variable dihydrogen content.

“Variable dihydrogen content” means according to the present invention that the mean dihydrogen concentration is not limited to a predefined fixed content whether in the gas stream obtained at the outlet of a method for producing or separating dihydrogen as described above, or in an initial stream before implementing the method for producing or separating dihydrogen.

“Real-time measurement” means, according to the present invention, that the measurement unit is capable of measuring the dihydrogen content present in the gas stream continuously and in real time.

“External receiver not burning dihydrogen” means according to the present invention a receiver different from a receiver of a combustion system or a unit intended to monitor combustion, in particular combustion parameters, of the gas stream comprising dihydrogen. Furthermore, the receiver is not comprised in the measurement and transmission system according to the invention.

The external receiver located outside the system according to the invention is a system for controlling a unit for producing dihydrogen, as defined above, or a unit for separating dihydrogen in a mixture based on natural gas and dihydrogen.

The measurement unit preferably comprises one of more means capable of measuring the dihydrogen content by spectroscopy.

Preferably, the measurement unit comprises one or more means for measuring the dihydrogen content by spectroscopy, having in particular an acquisition frequency less than 5 seconds, preferably less than 1 second.

Preferably, the measurement unit comprises one or more means capable of measuring the molar, mass or volume dihydrogen concentration in the gas stream by spectroscopy, having an acquisition frequency less than 5 seconds, more preferably an acquisition frequency less than 1 second.

The measurement unit comprises, preferably, one or more means for measuring the dihydrogen content in the gas stream by spectroscopy, in particular by Raman spectroscopy or laser beam absorption and wavelength modulation signal analysis spectroscopy.

In other words, the measurement means are in particular devices for measuring by spectroscopy, in particular by Raman spectroscopy or by laser beam absorption and wavelength modulation signal analysis spectroscopy.

Preferably, the measurement unit comprises one or more sensors capable of measuring the dihydrogen content in the gas stream by spectroscopy, more particularly by Raman spectroscopy or by laser beam absorption and wavelength modulation signal analysis spectroscopy.

The measurement unit may furthermore determine the concentration of other elements present in the gas stream, i.e. the concentration of elements different from dihydrogen from the unit for producing or separating dihydrogen.

Thus, the measurement unit can determine for example the content of natural gas not converted into dihydrogen by the unit for producing dihydrogen, for example the methane, butane or propane content, or the concentration of residues from natural gas decomposition or the ammonia cracking method.

Preferably, the measurement and transmission system according to the invention furthermore comprises one or more means for measuring the flow rate of the gas stream passing through said system.

The means for measuring the flow of the natural gas stream may be separate from or be an integral part of, preferably separate from, the measurement unit determining the dihydrogen content.

In other words, the means for measuring the flow rate of the gas stream may be included in a gas stream flow rate measurement unit separate from the measurement unit.

The means for measuring the flow rate may correspond to flow rate sensors, preferably mass or volume sensors, of the gas stream passing through said system.

Preferably, the means for measuring the flow rate are chosen in the group consisting of mechanical flow meters, thermal flow meters, differential pressure flow meters or ultrasound flow meters.

The means for measuring the flow rate may measure a mass or volume flow rate of the gas stream passing through said system, preferably with a precision uncertainty less than 1%, more preferably with an uncertainty less than 0.5%.

Preferably, the measurement and transmission system according to the invention comprises one or more means for measuring the mass flow rate of the gas stream passing through said system.

More preferably, the measurement and transmission system according to the invention comprises one or more Coriolis-effect mass flow meters and one or more thermal mass flow meters.

Preferably, the measurement and transmission system according to the invention comprises at least one measurement unit, as described above, and one or more means for measuring the flow rate of the gas stream passing through said system, preferably separate from said measurement unit.

Preferably, the measurement and transmission system according to the invention comprises:
at least one measurement unit, as described above, in particular containing one or more means for measuring in real time the mass dihydrogen content in the gas stream, and
one or more means for measuring the mass flow rate of the gas stream passing through said system, optionally separate from said measurement unit, in particular chosen in the group consisting of a Coriolis-effect mass flow meter or a thermal mass flow meter.

The processing and transmission unit makes it possible, on one hand, to process the information representative of the dihydrogen content measured by the measurement unit, as described above, and, on the other, to transmit said information to at least one external receiver located outside said system.

Preferably, the processing and transmission unit comprises one or more means capable of converting the dihydrogen content measured by the measurement unit, described above, into information representative of the dihydrogen content, in particular into a measurement signal representative of the dihydrogen content.

In particular, the unit may transmit the information to at least one external receiver not burning dihydrogen, in the form of a signal representative of the real-time measurement of the dihydrogen content in the gas stream.

More particularly, the unit may transmit said information, by wire or not, to at least one receiver different from a receiver of a combustion system or a unit intended to monitor the combustion parameter.

The information representative of the dihydrogen content corresponds preferably to the value of the dihydrogen concentration, in particular volume, mass or molar, in the gas stream, in particular from a unit for producing dihydrogen or separating dihydrogen.

Alternatively, the information representative of the dihydrogen content may correspond to information from which the dihydrogen content is capable of being inferred.

Processing of the information relating to the dihydrogen content may be performed digitally via a microprocessor and a computing algorithm.

The information representative of the dihydrogen content may be transmitted by wire or not, preferably wirelessly, for example by a radioelectric connection, to at least one system different from a combustion system or a unit intended to control the combustion parameters.

Advantageously, the processing and transmission unit furthermore makes it possible, on one hand, to process the information representative of the dihydrogen content measured by the measurement unit, as described above, and the information representative of the flow rate of the gas stream passing through the system according to the invention and, on the other, transmit said information to at least one system different from a combustion system or a unit intended to monitor combustion parameters.

In other words, preferably, the measurement unit comprises one or more sensors for measuring the dihydrogen content by spectroscopy and the processing and transmission unit comprises one or more means capable of converting the dihydrogen measurement into a signal representative of said content and transmitting said signal by wire or not, preferably wirelessly, for example by a radioelectric connection, to at least one system different from a combustion system or a unit intended to monitor combustion parameters.

The data item thus transmitted may correspond to the value of the measured quantity of dihydrogen contained in the gas stream and the value of the flow rate of the gas stream.

### Use of the System

The invention also relates to the use of the measurement and transmission system, as described above, to determine the dihydrogen content in a gas stream and transmit information representative of said dihydrogen content to at least one external receiver not burning dihydrogen.

Preferably, the system, as described above, is used in a dihydrogen production unit, in a natural gas station supplied by at least one dihydrogen production unit.

Preferably, the dihydrogen production unit may be an ammonia cracking unit, a natural gas steam reforming unit, with optionally carbon dioxide capture, a coal gasification unit, a natural gas decomposition unit or a water electrolysis unit.

Preferably, the dihydrogen production unit may be a natural gas steam reforming unit, with optional carbon dioxide capture, a natural gas decomposition unit or a water electrolysis dihydrogen production unit.

The external receiver is as defined above and corresponds to a system for controlling a unit for producing dihydrogen, as defined above, or a unit for separating dihydrogen in a mixture based on natural gas and dihydrogen.

Also advantageously, the system according to the invention may be used to control a system for producing dihydrogen (or a gas stream comprising a variable dihydrogen content) chosen in the group consisting of a natural gas decomposition system, an ammonia cracking system, a natural gas steam reforming system, a water electrolysis dihydrogen production system.

In particular, the natural gas decomposition system may be implemented by heating, in particular by plasma heating, induction heating, microwave heating, or by shock wave.

Also advantageously, the system according to the invention may be used to control a system for separating dihydrogen and natural gas, in particular a membrane or electrolysis separation system.

### Method

The present invention also relates to a method for real-time measurement of the dihydrogen content in a gas stream and for transmitting information representative of said dihydrogen content to at least one receiver not burning dihydrogen, comprising:
a step of distributing a gas stream comprising a variable dihydrogen content,
a step of measuring the variable dihydrogen content in the gas stream,
a step of processing and transmitting the information representative of said dihydrogen content determined during the measurement step to at least one receiver not burning dihydrogen.

Thus, the measurement step makes it possible to determine in real time the dihydrogen content present in a gas stream obtained at the outlet of a unit for producing dihydrogen or a unit for separating dihydrogen in a mixture based on natural gas and dihydrogen.

The dihydrogen present in the gas stream may be from a decarbonised or carbon, preferably decarbonised, source.

The dihydrogen may be from a carbon source, preferably capable of being obtained with a steam reforming method optionally comprising at least one step of capturing carbon dioxide.

Preferably, the dihydrogen is from a decarbonised source, preferably capable of being obtained with a water electrolysis method or an ammonia cracking method, more preferably with a water electrolysis method.

The step of measuring the dihydrogen content is preferably implemented by Raman spectroscopy or by laser beam absorption and wavelength modulation signal analysis spectroscopy.

The measurement step may in particular by implemented by one or more devices, in particular one or more sensors, for measuring by spectroscopy, in particular by Raman spectroscopy or by laser beam absorption and wavelength modulation signal analysis spectroscopy.

Advantageously, the method comprises a step of measuring the dihydrogen content in the gas stream, by spectroscopy and a step of measuring the flow rate of the gas stream.

The step of processing of the information representative of the dihydrogen content may be implemented by means of a microprocessor and a computing algorithm.

Preferably, the step of transmitting the information representative of said dihydrogen content to at least one receiver not burning dihydrogen is implemented by wire or by a radioelectric type connection.

Preferably, the external receiver may be chosen in the group consisting of a display system aimed at informing on the point-in-time dihydrogen content contained in the gas stream, a system for detecting dihydrogen leaks in a gas stream, or a system for controlling a method for producing dihydrogen or separating dihydrogen in a mixture based on natural gas and dihydrogen.

Advantageously, the external receiver is a system for controlling a method for producing dihydrogen or a method for separating dihydrogen in a mixture based on natural gas and dihydrogen.

Preferably, the external receiver is a system for controlling a unit for producing dihydrogen, as defined above, or a unit for separating dihydrogen in a mixture based on natural gas and dihydrogen, in particular by membrane or electrolysis.

Preferably, the method according to the invention is a method for optimising a method for producing dihydrogen, preferably chosen in the group consisting of natural gas decomposition, ammonia cracking, natural gas steam reforming or water electrolysis, or a method for separating a mixture of natural gas and dihydrogen, in particular a membrane or electrolysis separation method.

In particular, the method according to the invention comprises:
a step of distributing a gas stream comprising a variable dihydrogen content obtained by a system for producing dihydrogen or a system for separating a mixture of natural gas and dihydrogen,
a step of measuring the variable dihydrogen content present in the gas stream,
a step of processing and transmitting the information representative of said dihydrogen content determined during the measurement step to at least one system for controlling the system for producing dihydrogen or system for separating a mixture of natural gas and dihydrogen,
a step of controlling the system for producing dihydrogen or the system for separating a mixture of natural gas and dihydrogen according to the information received in respect of said dihydrogen content.

In particular, the step of measuring the dihydrogen content and the step of processing and transmitting the information representative of said dihydrogen content are implemented with the system according to the invention as described above.

The method according to the invention thus makes it possible to monitor one or more operating parameters of a unit for producing dihydrogen or a unit for separating a mixture of natural gas and dihydrogen, in particular the temperature, pressure conditions, electric field, flow rate of the stream at the inlet of the production and separation unit, according to the dihydrogen content information.

### Assembly

The invention also relates to an assembly comprising at least one system for supplying a gas stream containing a variable dihydrogen content, connected to at least one measurement and transmission system, and at least one system different from a dihydrogen burner.

Preferably, the system for supplying the gas stream comprises at least one unit for producing dihydrogen (or a stream comprising a variable dihydrogen content) or a unit for separating a mixture of natural gas and dihydrogen.

Preferably, the system for supplying the gas stream comprises at least one unit for distributing a gas or liquid stream, intended to be routed to at least one unit for producing dihydrogen or a unit for separating a mixture of natural gas and dihydrogen.

Preferably, the stream from the distribution unit is chosen in the group consisting of a stream comprising natural gas or a mixture of natural gas, a stream comprising ammonia, a stream comprising a mixture of natural gas and dihydrogen, or a liquid stream comprising water.

Preferably, the unit for producing dihydrogen (or a stream comprising a variable dihydrogen content) is chosen in the group consisting of a natural gas decomposition unit, a natural gas steam reforming unit optionally equipped with a system for capturing carbon dioxide, an ammonia cracking unit, or a water electrolysis dihydrogen production unit.

Preferably, the unit for producing dihydrogen (or a gas stream comprising a variable dihydrogen content) is chosen in the group consisting of a natural gas decomposition unit, an ammonia cracking unit, or a water electrolysis dihydrogen production unit.

Preferably, the unit for separating a mixture of natural gas and dihydrogen is a membrane or electrolysis separation unit.

Preferably, the unit for separating a mixture of natural gas and dihydrogen is a membrane, for example a membrane in polymer, ceramic form or in liquid form, separation unit.

Thus, the gas or liquid stream from the distribution unit is routed to the production or separation unit, as described above, to be transformed or converted into dihydrogen or a stream having a dihydrogen-enriched content.

In other words, the stream at the outlet of the production or separation unit, as described above, comprises an increased dihydrogen content with respect to the stream at the inlet of said production or separation unit.

The system different from a dihydrogen burner is in particular different from a combustion system or a unit intended to monitor combustion.

In particular, the system different from a dihydrogen burner is a system for controlling the dihydrogen production unit, as described above, or the unit for separating the mixture of natural gas and dihydrogen as defined above.

Thus, the system different from a dihydrogen burner receives the information representative of the dihydrogen content from the measurement and transmission system according to the invention.

Thus, the control system is in particular capable of controlling (or acting upon) one or more parameters of the unit for producing dihydrogen or the unit for separating the mixture of natural gas and dihydrogen, as defined above, to improve the transformation of the stream at the inlet of the unit for producing or separating dihydrogen according to said information representative of the dihydrogen content received.

In other words, the control system makes it possible in particular to enrich the dihydrogen stream at the outlet of the production or separation unit, as defined above, with dihydrogen with respect to the inlet stream by acting upon one or more parameters of the production or separation unit, for example the temperature, pressure conditions, the flow rate of the stream at the inlet of said units, the electric field induced.

Other features and advantages of the invention will become apparent following the detailed examination of an embodiment taken by way of non-limiting example of a measurement and transmission system according to the invention and illustrated by the appended drawing.

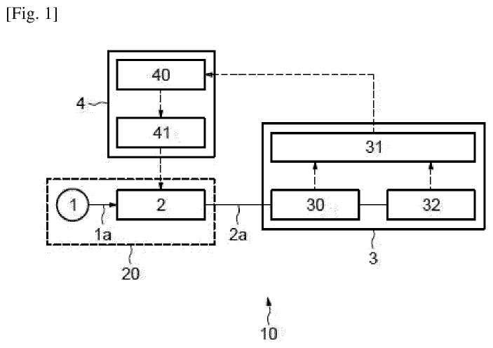

In FIG. 1, an assembly 10 is represented, comprising a unit 1 for distributing a gas or liquid stream, a unit 2 for producing a stream containing a variable dihydrogen content, a system 3 for measuring the dihydrogen content and transmitting the information representative of said content to a system 4 different from a dihydrogen burner.

The distribution unit 1 routes a gas or liquid stream via at least one connection circuit 1a to the production unit 2 such that the stream is processed to be transformed or converted at least in part into dihydrogen.

The stream thus routed may be a stream comprising natural gas or a mixture of natural gas, a stream comprising ammonia, a stream comprising a mixture of natural gas and dihydrogen, or a liquid stream comprising water.

The production unit 2 is connected to the measurement and transmission system 3 via a connection circuit 2a. In this way, the stream comprising a variable dihydrogen content is routed to the system 3.

As stated above, the production unit 2 makes it possible to transform or convert at least a portion of the inlet stream into a gas stream comprising a dihydrogen-enriched content.

In other words, the dihydrogen content in the outlet stream of the unit 2 is greater than the dihydrogen content present in the inlet stream.

Coupling the distribution unit 1 and the production unit 2 may form a system 20 for distributing a gas stream containing a variable dihydrogen content.

The system 3 comprises at least one unit 30 for measuring the variable dihydrogen content present in the gas stream from the production unit 2, and at least one unit 31 for processing and transmitting the information representative of said dihydrogen content determined by said measurement unit 30.

The measurement unit 30 determines in real time the dihydrogen content present in the gas stream from the dihydrogen production unit 2.

The unit 30 may comprise one or more sensors for measuring the dihydrogen content by spectroscopy, preferably by Raman spectroscopy.

The unit 31, on one hand, digitally processes the information representative of the dihydrogen content measured by the measurement unit 30 and, on the other, transmits said information, by wire or radioelectrically, to the system 4.

The system 3 may furthermore comprise a unit 32 for measuring the flow rate of the gas stream passing through the system.

The measurement unit 32 may comprise several flow rate sensors, for example gas stream mass sensors.

The system 4 comprises a receiver 40 of the information representative of the dihydrogen content measured by the measurement unit 30.

The system 4 also comprises a unit 41 capable of controlling the dihydrogen production unit 2, in particular one or more operating parameters of the dihydrogen production unit 2, to improve the dihydrogen conversion rate.

Thus, according to the information sent by the system 3 on the dihydrogen content, the unit 41 of the system 4 controls one or more control parameters of the method for producing dihydrogen implemented in the unit 2 in a closed loop to optimise the dihydrogen concentration in the gas stream at the outlet of the unit 2.

The production unit 2 may preferably be a unit for decomposing a stream comprising natural gas or a natural gas mixture, a unit for steam reforming of a stream comprising natural gas or a natural gas mixture, a unit for cracking a stream comprising ammonia or a water electrolysis unit.

Alternatively, in FIG. 1, the unit 2 may be a unit for separating a mixture of natural gas and dihydrogen.

In this case, the unit 2 is a unit for separating a mixture of natural gas and dihydrogen by membrane or by electrolysis.

Thus, in this case, the unit 1 distributes a stream comprising a mixture of natural gas and dihydrogen and the unit 2 aims to separate, for example via a membrane or an electrolysis, the mixture of natural gas and dihydrogen to recover a stream containing a dihydrogen content.

Coupling the distribution unit 1 and the separation unit 2 may form a system 20 for distributing a gas stream comprising a variable dihydrogen content.

The measurement unit 30 determines in real time the dihydrogen content present in the gas stream from the unit 2 for separating the mixture of natural and dihydrogen.

According to the information sent by the unit 31 of the system 3 on the dihydrogen content, the unit 41 of the system 4 controls one or more control parameters of the method for separating the mixture of natural gas and dihydrogen in a closed loop to improve the dihydrogen recovery rate.

Thus, the system 4 may act upon and/or control the membrane or electrolysis separation method implemented by the separation unit 2.

The system 3 according to the invention advantageously makes it possible to be able quantify dihydrogen drawn from a natural gas and dihydrogen network in order to supply a dihydrogen distribution station.

Thus, the system 3 makes it possible to measure and share in real time the point-in-time dihydrogen content present in the gas stream passing through the assembly 10.

1. System for real-time measurement of a dihydrogen content present in a gas stream and for transmitting information representative of said dihydrogen content to at least one external receiver (4) located outside said system; said system (3) comprising:
at least one unit (30) for measuring the dihydrogen content contained in the natural gas stream passing through said system,
at least one unit (31) for processing and transmitting the information representative of said dihydrogen content determined by said measurement unit (30) to at least one external receiver (4) located outside said system not burning dihydrogen,
said receiver (4) corresponding to a system for controlling a unit for producing dihydrogen or a unit for separating dihydrogen in a mixture based on natural gas and dihydrogen.

2. System according to claim 1, wherein the measurement unit (30) comprises one or more means for measuring the dihydrogen content present in the gas stream, having an acquisition frequency less than 5 seconds.

3. System according to claim 1, wherein the measurement unit (30) comprises one or more means for measuring the dihydrogen content in the gas stream implemented by spectroscopy, preferably by Raman spectroscopy or by laser beam absorption and wavelength modulation signal analysis spectroscopy.

4. System according to claim 1, further comprising at least one unit (32) for measuring the flow rate of the gas stream passing through said system (3).

5. The system according to claim 1, wherein said receiver (4) is not burning dihydrogen, or is in a unit for producing dihydrogen, or is in a natural gas station supplied by at least one unit for producing dihydrogen; said receiver (4) corresponding to a system for controlling a unit for producing dihydrogen or a unit for separating dihydrogen in a mixture based on natural gas and dihydrogen.

6. Method for real-time measurement of the dihydrogen content present in a gas stream and for transmitting information representative of said dihydrogen content to at least one receiver not burning dihydrogen, comprising:
a step of distributing a gas stream comprising a variable dihydrogen content, obtained by a unit (2) for producing dihydrogen or a unit (2) for separating a mixture of natural gas and dihydrogen,
a step of measuring the dihydrogen content present in the gas stream,
a step of processing and transmitting the information representative of said dihydrogen content determined during the measurement step to at least one receiver (4) not burning dihydrogen; said receiver (4) corresponding to a system for controlling a unit (2) for producing dihydrogen or a unit (2) for separating dihydrogen in a mixture based on natural gas and dihydrogen.

7. Method according to claim 6, wherein the dihydrogen present in the gas stream is from a decarbonised source.

8. Method according to claim 6, wherein the dihydrogen present in the gas stream is from a carbon source.

9. Method according to claim 6, wherein the dihydrogen present in the gas stream is from a separation method in a mixture comprising natural gas and dihydrogen.

10. Method according to claim 6, further comprising:
a step of distributing a gas stream comprising a variable dihydrogen content obtained by a system (2) for producing dihydrogen or a system (2) for separating a mixture of natural gas and dihydrogen,
a step of measuring the variable dihydrogen content present in the gas stream,
a step of processing and transmitting the information representative of said dihydrogen content determined during the measurement step to at least one system (4) for controlling the system (2) for producing dihydrogen or system (2) for separating a mixture of natural gas and dihydrogen,
a step of controlling the system (2) for producing dihydrogen or the system (2) for separating a mixture of natural gas and dihydrogen according to the information received of said dihydrogen content.

11. Assembly (10) comprising:
at least one system (20) for supplying a gas stream containing a variable dihydrogen content, comprising at least one unit (1) for distributing a gas or liquid stream, connected to at least a unit (2) for producing dihydrogen or a unit (2) for separating a mixture of natural gas and dihydrogen, connected to at least one measurement and transmission system (3), as defined according to claim 1, and
at least one system (4) different from a dihydrogen burner; the system (4) being a system for controlling a unit (2) for producing dihydrogen or a system for controlling a unit (2) for separating the mixture of natural gas and dihydrogen.

12. Assembly (10) according to claim 11, wherein the unit for producing dihydrogen is chosen in the group consisting of a natural gas decomposition unit, a natural gas steam reforming unit optionally equipped with a system for capturing carbon dioxide, an ammonia cracking unit, or a water electrolysis dihydrogen production unit.

13. Assembly (10) according to claim 11, wherein the unit for separating a mixture of natural gas and dihydrogen is a membrane or electrolysis separation unit.

14. Assembly (10) according to claim 11, wherein system (4) is a system for controlling the unit (2) for producing dihydrogen, as defined according to claim 11, or a system for controlling the unit (2) for separating the mixture of natural gas and dihydrogen, as defined according to claim 11.
